(19) United States
(12) Patent Application Publication
(10) Pub. No.: US 2023/0255719 A1
(43) Pub. Date: Aug. 17, 2023

(54) DEVICE AND METHOD FOR IMAGING VASCULATURE

(71) Applicant: VENA MEDICAL HOLDINGS CORP., Kitchener (CA)

(72) Inventors: Michael PHILLIPS, Plumweseep (CA); Phillip COOPER, Kitchener (CA)

(21) Appl. No.: 18/138,971

(22) Filed: Apr. 25, 2023

Related U.S. Application Data
(62) Division of application No. 16/638,380, filed on Feb. 11, 2020, now abandoned.
(60) Provisional application No. 62/547,317, filed on Aug. 18, 2017.

(51) Int. Cl. A61B 90/00; A61B 5/00; A61B 5/06; A61B 1/00; A61B 1/04; A61B 1/06

(52) U.S. Cl. CPC A61B 90/361; A61B 5/0066; A61B 5/0086; A61B 5/065; A61B 5/6876; A61B 1/000094; A61B 1/044; A61B 1/046; A61B 1/0605; A61B 2090/306

(57) ABSTRACT

A device and method for imaging vasculature are provided. The device includes an imaging probe to be inserted into a vasculature. The imaging probe emits infrared light through blood toward the vasculature, and gathers reflected from the vasculature for imaging. The device includes an infrared light source optically coupled to the imaging probe to provide infrared light, and an infrared light detector optically to the imaging probe to generate an imaging signal from the reflected light that is gathered. The device further includes a controller coupled to the infrared light source and coupled to the infrared light detector to generate an image of the vasculature from the imaging signal. The controller may employ ballistic photon imaging techniques, gated imaging techniques, polarizing light imaging techniques, structured light imaging techniques, and the like.

### CROSS-REFERENCE TO RELATED APPLICATIONS

This application is a divisional of U.S. patent application Ser. No. 16/638,380. which claims priority to U.S. Provisional Patent Application No. 62/547,317, filed Aug. 18, 2017, the entirety of each of which is incorporated herein by reference.

### FIELD

The present disclosure relates to interventional medicine, and in particular to the imaging of vasculature.

### BACKGROUND

Interventional medicine is a medical specialty where physicians, such as interventional cardiologists, interventional radiologists, interventional neuroradiologists and endovascular neurosurgeons perform minimally invasive procedures using image-guided technologies. Physicians may employ image-guided techniques to navigate medical devices through vasculature to reach a target location where a medical procedure is to be performed.

### SUMMARY

According to an aspect of the specification, an imaging device is provided. The imaging device includes an imaging probe having a proximal end and a distal end, the distal end for insertion into a vasculature. The imaging probe is to emit infrared light from the distal end of the imaging probe toward the vasculature through blood, gather reflected light comprising at least a portion of the infrared light reflected from the vasculature through the blood, and transmit the reflected light to the proximal end of the imaging probe. The imaging device further includes an infrared light source optically coupled to the proximal end of the imaging probe to provide the infrared light to the imaging probe for emission toward the vasculature. The imaging device further includes an infrared light detector optically coupled to the proximal end of the imaging probe to receive the reflected light from the imaging probe to generate an imaging signal from the reflected light. The imaging device further includes a controller coupled to the infrared light source and coupled to the infrared light detector to generate an image of the vasculature from the imaging signal.

According to another aspect of the specification, a method for imaging vasculature is provided. The method includes emitting infrared light from an imaging probe toward vasculature through blood, gathering reflected light from the vasculature through the blood, generating an imaging signal from the reflected light, and generating an image of the vasculature from the imaging signal.

### BRIEF DESCRIPTION OF THE DRAWINGS

FIG. 1 depicts a schematic diagram of an example device for imaging vasculature, and further depicts a perspective view of an example distal end of an example imaging probe, with portions broken away.

FIG. 2 depicts a perspective view of the distal end of the imaging probe of the device of FIG. 1.

FIG. 3 depicts a schematic diagram of another example device for imaging vasculature, and further depicts an enlarged cross-sectional view of another example proximal end of another example imaging probe.

FIG. 4 depicts a schematic diagram of the proximal end of the imaging probe of FIG. 3 decoupled from an infrared light source and infrared light detector of the device of FIG. 3.

FIG. 5 depicts a plan view of an example sheathing for an example imaging guidewire.

FIG. 6 depicts a schematic diagram of another example device for imaging vasculature, and further depicts a perspective view of another example distal end of another example imaging probe, with portions broken away.

FIG. 7 depicts a schematic diagram of an example angle gate filter at a distal end of another example imaging probe.

FIG. 8 is a flowchart of an example method for imaging vasculature.

FIGS. 9A and 9B are schematic diagrams of an example coherent fiber bundle angle gate filter.

### DETAILED DESCRIPTION

Physicians performing interventional procedures may use image-guided technologies to assist with positioning a medical device at an appropriate location within a patient's vasculature. Typically, a medical device may be brought to an appropriate location by a physician inserting a guidewire followed by a catheter or microcatheter. A common image-guided technique to ensure that a device reaches a target location is to steer the guidewire and catheters through the patient's vasculature using fluoroscopy. Fluoroscopy is pulse-based x-ray imaging that requires the use of a contrast agent. The contrast agent is typically administered through a catheter or microcatheter and allows the physician to visualize the vasculature on an x-ray image. Once the interventionalist has gained access to the appropriate location in the vasculature, they can perform a variety of procedures. These procedures may include angioplasties in which inflatable balloons and stents are used to widen narrow or obstructed arteries or veins, embolization procedures used to cause ischemia of lesions by restricting the flow of blood to tumors or fibroids, or thrombectomy procedures where stent-based retrieval tools and aspiration catheters are used to remove blood clots in stroke treatment.

While standard fluoroscopy techniques for intravascular navigation may be sufficient for procedures within routine vasculature, fluoroscopy-based navigation techniques are inadequate for navigating in tortuous anatomy. The two-dimensional nature of the fluoroscopy x-ray may not afford sufficient perspective to accurately determine the angle at which bifurcations occur. Fluoroscopy also may not allow physicians to accurately see tiny endovascular devices such as small stents and coils used in cerebrovascular interventions. In neuro interventions, precise placement of small and expensive devices is critical for both patient outcomes and the reduction of healthcare costs by limiting extra device usage. Fluoroscopy also may not allow physicians to accurately characterize soft tissue such as thrombi or emboli. For example, in endovascular stroke treatment, fluoroscopy may not provide the physician with information regarding qualities of the clot, such as whether the clot is soft, recently formed, hard, or calcified. This deficiency may impact device selection in the treatment algorithm. Selecting the wrong device may increase treatment time and decrease success rates. Further, since fluoroscopy does not convey information about such characteristics of a clot, or the position of the clot, a physician using fluoroscopy may also be unable to determine whether the clot is successfully being removed by a retrieval tool until the tools are completely pulled out of the body due to the lack of visualization. Finally, fluoroscopy may not be able to reliably confirm whether complete revascularization of the afflicted vessel was achieved, or whether all of the clot was removed.

Moreover, fluoroscopy techniques necessarily involve the use of contrast agents such as iodine, which may also pose problems in the interventional suite since the contrast agents may be prone to adhere to operative tools and disrupt workflow. Contrast agents may also be nephrotoxic, which is detrimental for patients with weak or impaired kidney function such as small children and the elderly, limiting the candidate pool for minimally invasive procedures. Another problem with fluoroscopy is risks associated with the application of radiation. Interventional physicians, nurses and technicians who perform these procedures are subjected to x-ray radiation at high levels for long intervals, and typically receive three to ten times the annual radiation dose of the average citizen. Varying studies attempt to quantify the elevated instances of several types of cancers, as well as other afflictions like cataracts, in healthcare workers exposed to x-ray radiation. To mitigate radiation exposure, individuals working around fluoroscopy may be required to wear lead vests, aprons and thyroid shields; often for upwards of eight hours a day. Heavy lead is uncomfortable and unergonomic. Over the course of their careers, over half of interventional physicians report orthopedic problems that could be at least partially attributed to lead vests. The patient also receives varying amounts of radiation which can limit maximum procedure times for small children and in some cases, can cause radiation burns.

As an augmentation to standard fluoroscopy techniques, computerized tomography (CT) scans and cone beam computerized tomography (CBCT) scans may be employed. These techniques involve compiling axial image slices of the patient's body and recreating a three-dimensional model of the patient's anatomy. When used with fluoroscopy, a three-dimensional visualization of the patient's vasculature may be created for guidewire navigation. Unfortunately, the patient must remain still and hold their breath while the scan is occurring. Once the scan is complete, the virtual model of the vasculature is static and is instantly outdated due to movement caused by the patient's breathing. The CT or CBCT generated image is not real-time and is unable to show the tip of a guidewire as it is advanced through patient vasculature. The lack of real-time imaging renders such techniques incapable of assisting in interventions, such as stent placement, angioplasties or thrombectomies. Fluoroscopy techniques, even when augmented by CT or CBCT techniques, do not provide a view from inside a patient's blood vessel.

Some technologies, such as optical coherence tomography (OCT) and intravascular ultrasound (IVUS) may provide a view from inside a patient's blood vessels. These technologies, however, are diagnostic tools that are not useful in guidewire navigation. Both OCT and IVUS are catheter-based units that must be advanced and pulled back as a probe scans and rotates inside of an artery or vein. Thus, these technologies do not provide forward viewing, nor do they provide real-time viewing—two beneficial attributes of an intravascular navigational imaging device. For example, the lack of real-time imaging precludes their use in assisting aneurysm coiling, thrombectomies, and stent placement.

Other techniques which provide a view from inside a patient's blood vessel include angioscopes. Angioscopes are visible light endoscopes that are inserted into blood vessels for the purpose of intravascular imaging. Angioscopes, whether coherent fiber bundle (CFB) or scanning fiber endoscopy (SFE) based, are not suitable for navigation through blood vessels containing blood because blood is opaque to visible light. Angioscopes may be used to provide a view from inside a patient's blood vessel by inflating a catheter-based balloon inside the blood vessel to temporarily occlude a blood vessel, flushing the blood vessel with clear saline, and taking images using the angioscope before retrograde blood flow begins. Such techniques may be appropriate for diagnostic or inspection purposes, but not for navigation purposes since the catheter may not be advanced with the inflated balloon deployed.

A device for imaging vascular may be provided, which includes an imaging probe to be inserted into a vasculature and to emit infrared light through blood toward the vasculature and to gather reflected from the vasculature for imaging. The imaging device further includes an infrared light source optically coupled to the imaging probe for emission toward the vasculature. The imaging device further includes an infrared light detector optically coupled to the imaging probe to generate an imaging signal from the reflected light, and a controller coupled to the infrared light source and coupled to the infrared light detector to generate an image of the vasculature from the imaging signal. The controller may employ a number of imaging techniques, including ballistic photon imaging techniques, gated imaging techniques, polarizing light imaging techniques, structured light imaging techniques, and the like. The imaging probe may include pushable and trackable sheathing to be navigated through a catheter. The imaging probe may also include guidewire sheathing for self-navigation through the vasculature. The imaging probe may also be included as part of another catheter device. Thus, interventional medical procedures may be guided by an imaging device which provides imagery from the inside of a blood-filled vasculature. A physician using such an imaging device may therefore view the features of a vasculature from inside the vasculature, through blood, and in real time, to assist with navigating through the vasculature.

Further, a method for imaging vascular may be provided, which includes emitting infrared light from an imaging probe toward the vasculature through blood, gathering reflected light from the vasculature through the blood, generating an imaging signal from the reflected light, and generating an image of the vasculature from the imaging signal. The image may be generated according to a ballistic imaging, gated imaging, structured light, polarizing light imaging technique, and the like.

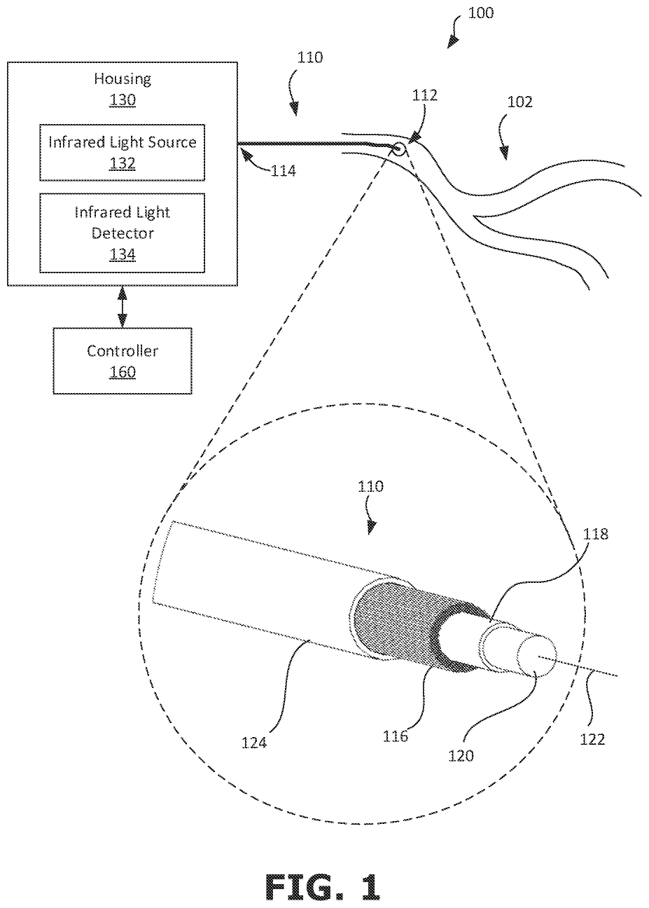

FIG. 1 depicts an example device 100 for imaging vasculature. The device 100 includes an imaging probe 110 which is navigable through vasculature 102. The imaging probe 110 includes a proximal end 114 and a distal end 112 opposite the proximal end 114. The distal end 112 is configured for insertion into vasculature 102, and the proximal end 114 is configured for connection with an infrared light source 132 and an infrared light detector 134, which may be disposed in a housing 130. In some examples, the vasculature 102 may include a blood-filled vasculature of a patient.

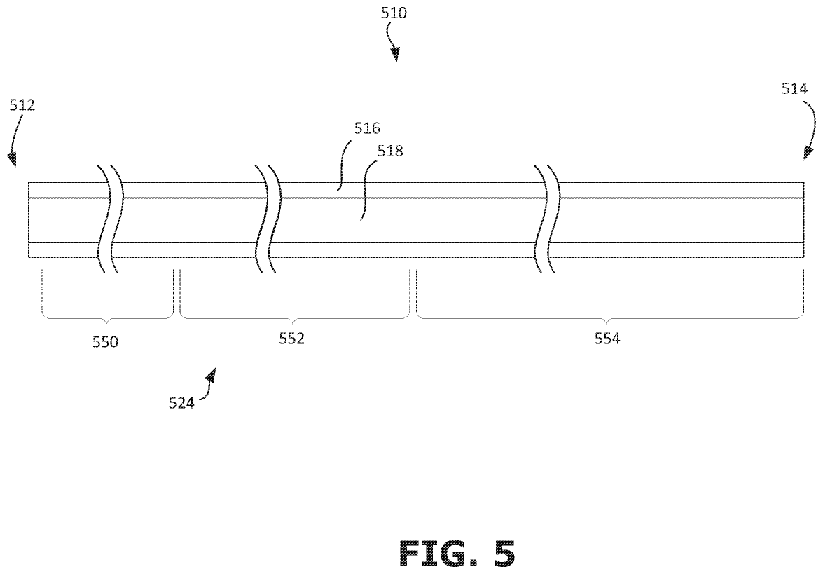

In some examples, the imaging probe 110 may be pushable through a catheter, and trackable through a catheter. In some examples, the imaging probe 110 may include a scanning fiber endoscope. In some examples, the imaging probe 110 may be incorporated into a catheter. In some examples, the imaging probe 110 may include a guidewire sheathing, as discussed in greater detail below with reference to FIG. 5.

The imaging probe 110 may be configured for insertion into the vasculature 102, and navigation through the vasculature 102, by being dimensioned accordingly. In examples in which the imaging probe 110 is incorporated into a catheter, the imaging probe 110 may have a diameter between about 1.5 Fr (French Gauge) to about 3 Fr. In examples in which the imaging probe 110 includes a guidewire sheathing, the imaging probe 110 may have a diameter between about 14 thousandths of an inch (0.014″) and about 38 thousandths of an inch (0.038″). In examples in which the imaging probe 110 includes a scanning fiber endoscope, the imaging probe 110 may have a diameter between about 3 Fr to about 5 Fr. Generally, in some examples, the imaging probe 110 may have a diameter, between about 1 Fr and about 12 Fr, or in some examples, between about 1.5 Fr to about 3 Fr. The imaging probe 110 may further be configured to have a suitable bending radius according to the size of the vasculature being navigated. For example, for navigation through tortuous vasculature, the imaging probe 110 may have a bending radius between about 2.5 mm and about 5 mm. The imaging probe 110 may have a larger bending radius for navigation through larger vasculature. It is to be understood that such dimensions are examples, and other suitable dimensions are also contemplated.

The imaging probe 110 includes a bundle of illuminating optical fibers 116 to transmit infrared light from the distal end 112 of the imaging probe 110 to the vasculature 102 through blood. The imaging probe 110 further includes a bundle of imaging optical fibers 118 to gather reflected light, the reflected light including at least a portion of the infrared light reflected from the vasculature 102 through the blood. The bundle of imaging optical fibers 118 is further to transmit the reflected light to the proximal end 114 of the imaging probe 110 for imaging. The bundles 116, 118, each extend from the proximal end 114 to the distal end 112 of the imaging probe 110. The fibers in the bundles 116, 118, may include polymer fibers, glass fibers, or any other suitable optical fiber.

The infrared light source is to provide the infrared light to the bundle of illuminating optical fibers 116 for transmission to the vasculature 102. The infrared light detector 134 may include an infrared camera or another imaging sensor to receive the reflected light from the bundle of imaging optical fibers 118 and to generate an imaging signal from the reflected light.

The device 100 further includes a controller 160 coupled to the infrared light source 132 and the infrared light detector 134. The controller 160 processes imaging signals captured by the infrared light detector 134 to generate images of the vasculature 602. The controller 160 may include any quantity and combination of a processor, a central processing units (CPU), a microprocessor, a microcontroller, a field-programmable gate array (FPGA), and similar, a memory storage unit including volatile and/or non-volatile storage, and a network interface for communication via one or more computing networks. The controller 160 may comprise, or may be connected to, a computing device such as a laptop computer, desktop computer, smartphone, remote server, and the like. The controller 160 may be housed in the housing 130, or may be external to the housing 130.

The controller 160 may control the infrared light source 132 to provide infrared light including wavelengths which may suitably penetrate through blood for imaging purposes, namely, any one of, or any combination of, 1050 nm, 1300 nm, 1550 nm, and 1650 nm. The controller 160 may also control the infrared light source 132 to provide a wavelength distribution centered at one or more of these wavelengths.

Further, the controller 160 may control the infrared light source 132 and the infrared light detector 134 according to an imaging process to generate the image of the vasculature 102. The imaging process may be based on selecting only a portion of reflected photons reflected off the vasculature 102 to be used for imaging purposes. For example, the imaging process may include selecting or excluding ballistic photons, snake photons, and scattered photons from imaging. The imaging process may include ballistic photon imaging, gated imaging, structured light imaging, polarizing imaging, and the like.

In a ballistic photon imaging process, the device 100 may further include an angle gate filter at the distal end of the imaging probe 110 to filter the reflected light to remove scattered photons from the reflected light, and the controller 160 may control the infrared light source 132 and the infrared light detector 134 to generate an image according to a ballistic photon imaging process. Thus, scattered photons and some snake photons may be filtered and excluded from imaging. Input photons which return at least partly scattered, such as scattered photons and snake photons, are likely to reduce the quality of an image of vasculature generated thereby, whereas ballistic photons may provide clear light for imaging purposes.

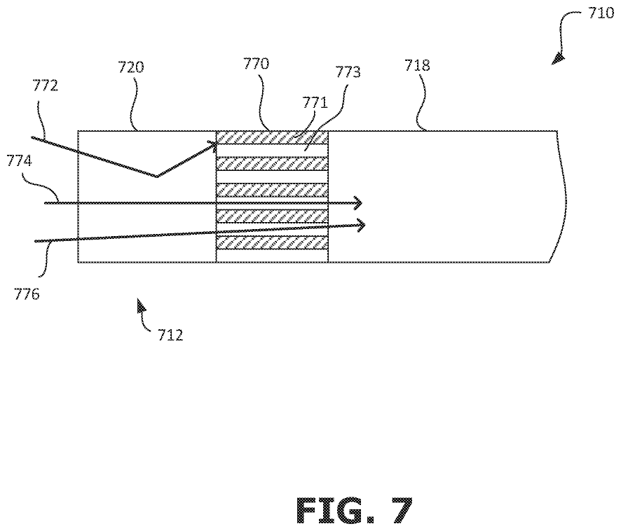

With reference to FIG. 7, for example, an angle gate filter 770 is situated between a lens 720 and a bundle of imaging optical fibers 718, coupled to a distal end 712 of an imaging probe 710. The angle gate filter 770 includes walls 771 to absorb or block scattered photons 772 and channels 773 to permit ballistic photons 774 through, thereby filtering the reflected light. Thus, an image of vasculature which is not distorted by scattered photons may be generated. In some examples, a portion of snake photons 776 may be permitted through. It is also contemplated that a disposed film on the lens 720 may similarly filter scattered photons and a portion of snake photons.

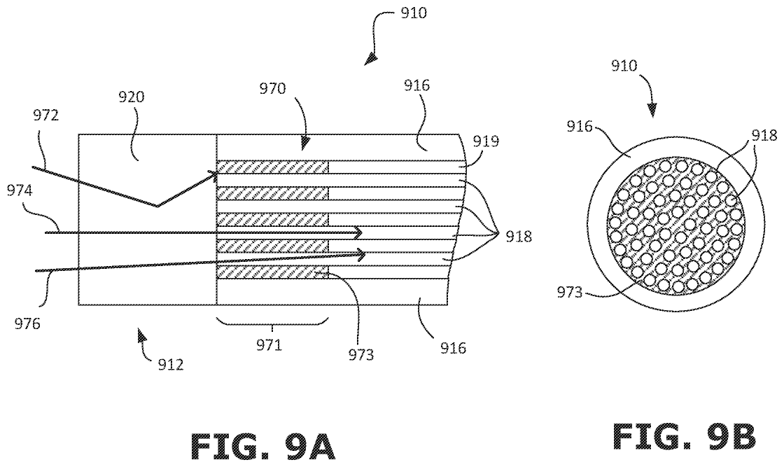

As another example, with reference to FIGS. 9A and 9B, a coherent fiber bundle angle gate filter 970 is situated at a distal end 912 of an imaging probe 910. The imaging probe 910 includes a bundle of illuminating optical fibers 916, a bundle of imaging optical fibers 918, and a lens 920 at the distal end 912 of the imaging probe 910. The imaging optical fibers 918 have spaces 919 between individual optical fibers. Along a filtering portion 971 of the imaging probe 910, the spaces 919 are packed with a filtering material 973 such as an ethylene methyl acrylate cladding. The filtering material absorbs or blocks scattered photons 972, allowing ballistic photons 974 and a portion of snake photons 976 to pass through the bundle of imaging optical fibers 918. Thus, scattered photons and a portion of snake photons may be filtered from the reflected light used for imaging so that a clearer image of vasculature may be generated.

Continuing with reference to FIG. 1, in a gated imaging process, the infrared light detector 134 may include an infrared camera having a shutter, and the controller 160 may control the infrared light source 132 and the infrared camera to generate an image according to a gated imaging process. The controller 160 may thus control the transmission of infrared light from the infrared light source 132, and simultaneously control the shutter to control exposure of the infrared camera such that the infrared camera excludes photons which have been scattered from imaging while receiving ballistic photons for imaging. In other words, the infrared light source 132 and the shutter of the infrared camera may be synchronized to block scattered photons. A distribution of reflected photons gathered and imaged by the infrared camera may indicate that ballistic photons tend to be gathered earliest, followed by snake photons, followed by scattered photons. Thus, after a pulse of infrared light is transmitted to the vasculature 102, the infrared camera may be configured to use only the photons captured during a pre-determined interval expected to correspond to ballistic photons, or a distribution of ballistic and snake photons, to the exclusion of scattered photons.

In a structured light imaging process, the infrared light source 132 may include a pattern-generating light source to project a pattern, such as a grid, or shapes, onto the vasculature 102 for use in generating a 3-dimensional representation of the vasculature 102. The bundle of illuminating optical fibers 116 may further include coherent illuminating optical fibers to emit the infrared light, including the pattern for projection onto the vasculature 102. Thus, the controller 160 may control the infrared light source 132 and the infrared light detector 134 to generate an image according to a structured light imaging process. In some examples, in operation, the imaging probe 110 may be advanced, retracted, or otherwise moved through the vasculature 102, as infrared light is emitted toward the vasculature 102. In some examples, a plurality of pulses of infrared light are emitted toward the vasculature 102. In some of such examples, gated imaging or ballistic imaging techniques may also be employed, whereby scattered reflected photons are excluded from the reflected light which is processed. Reflected light from the infrared light may be collected by the imaging probe 110 to generate successive imaging signals and/or images. The images may be successively generated based on the pulses of infrared light, shutter timing, or another technique for segmenting images. The successive images may be assembled by the controller 160 to generate a three-dimensional model of the vasculature 102. Thus, the controller 160 may control the infrared light source 132 and the infrared light detector 134 to generate a three-dimensional model of the vasculature 102 according to a structured light imaging process.

In a polarizing imaging process, the device 100 may further include a polarization filter to filter the reflected light to remove polarized light from the reflected light, and the controller 160 may be configured to control the infrared light source 132 and the infrared light detector 134 to generate an image according to a polarizing light imaging process. In particular, the controller 160 may be configured to control the infrared light source 132 to provide polarized infrared light to the bundle of illuminating optical fibers 116. The polarization of light that is scattered by blood in the vasculature 102 changes, whereas the polarization of light reflected from by the vasculature 102 remains the same. A polarization filter may be located between the distal end 112 and the proximal end 114 of the imaging probe 110. In some examples, a polarization filter may be located at the distal end 112 of the imaging probe 110 to inhibit polarized light from travelling down the imaging probe 110. In other examples, a polarization filter may be located at the proximal end 114 of the imaging probe 110, or in the housing 130. In some examples, a polarization filter may include a film disposed on the lens 120, or a filter optically coupled to the distal end 112 of the imaging probe 110 between the lens 120 and the bundles 116, 118.

The imaging probe 110 further includes a conduit 124, extending from the proximal end 114 to the distal end 112, to contain the illuminating optical fibers 116 and the imaging optical fibers 118.

In some examples, the conduit 124 may include a cord, tube, flexible shaft, coil and braid, or other structure for allowing the imaging probe 110 to be navigable through the vasculature 102. Thus, the conduit 124 may include a pushable and trackable sheathing to enable pushing and tracking of the imaging probe 110 through a catheter. In some examples, the conduit 124 may include an inner wall of a catheter in which the imaging probe 110 is incorporated.

In some examples, the conduit 124 may include a guidewire sheathing for navigating the imaging probe 110 through the vasculature 102. A guidewire sheathing may enable the imaging probe 110 to be steerable, shapeable, and torquable to facilitate navigation through the vasculature 102. A guidewire sheathing is discussed in greater detail with respect to FIG. 5 below.

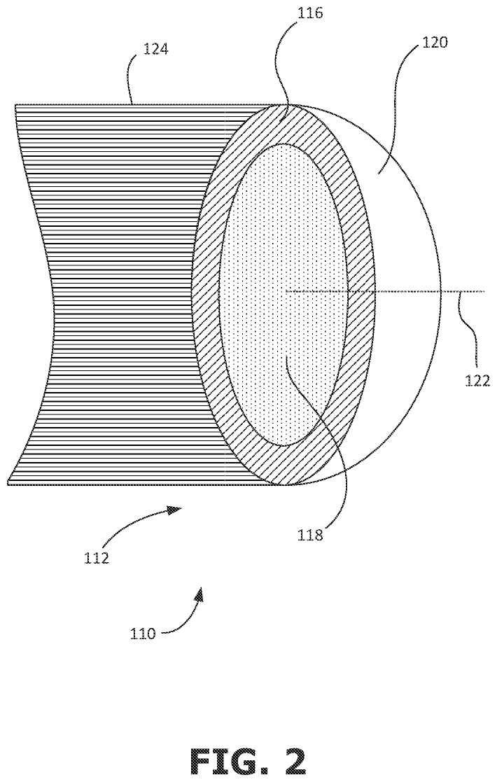

FIG. 2 depicts a perspective view of the distal end 112 of the imaging probe 110 of the device 100. With reference to FIG. 2, and with continued reference to FIG. 1, it may be seen that the imaging probe 110 further includes optics at the distal end 112 to collect reflected light to be gathered by the bundle of imaging optical fibers 118. For example, the optics may include a lens 120, such as a micro lens or a graded-index (GRIN) lens, optically coupled to the distal end 112 of the imaging probe 110.

Further, with reference to FIG. 2, it may be seen that in the present example the imaging probe 110 defines a longitudinal axis 122, and that the bundle of imaging optical fibers 118 extends along the longitudinal axis 122, and that the bundle of illuminating optical fibers 116 is arranged in a ring around the bundle of imaging optical fibers 118. In other words, the bundle of imaging optical fibers 118 are arranged in a circle, the bundle of illuminating optical fibers 116 are arranged in a ring around the circle, and the circle and ring are about concentric about a longitudinal axis 122, which is to be oriented toward the vasculature 102 being imaged.

In other examples, the bundle of imaging optical fibers 118 may be arranged in a ring around the bundle of illuminating optical fibers 116, and the lens 120 may include a ringed lens. In some examples, the imaging optical fibers 118 may be rotatable about the longitudinal axis 122, as in a scanning fiber endoscope.

The optics may further include a filter, such as an angle gate filter, which may remove scattered light or undesired wavelengths of light from collection by the bundle of imaging optical fibers 118 or a polarization filter to filter polarized light from the reflected light.

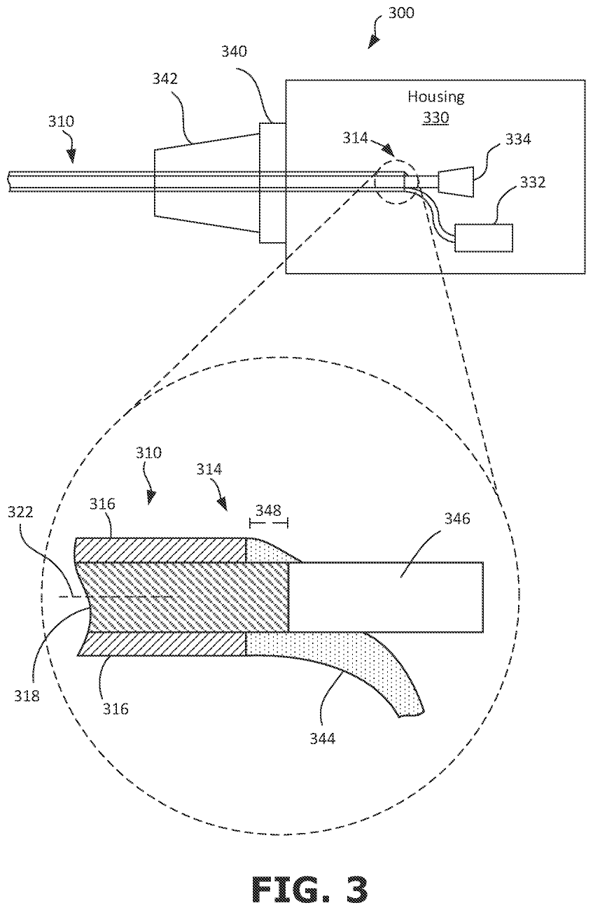

FIG. 3 depicts a schematic diagram of another example device 300 for imaging vasculature. The device 300 is substantially similar to the device 100 with like components having like numbers, however in a “300” series rather than a “100” series. With reference to FIG. 3, the device 300 hence includes an imaging probe 310 having a proximal end 314, a bundle of illuminating optical fibers 316 and a bundle of imaging optical fibers 318. The device 300 further includes an infrared light source 332 and an infrared light detector 334 which may be disposed in a housing 330. Although not shown, it is to be understood that the device 300 includes a controller similar to the controller 160. For further description of the above elements of the device 300, the description of the device 100 of FIG. 1 may be referenced. For sake of clarity, only the differences between the device 300 and the device 100 will be described in detail.

In contrast to the device 100, the device 300 further includes a coupling mechanism to reversibly and rotatably couple the proximal end 314 of the imaging probe 310 to the housing 330 and to the infrared light source 332 and infrared light detector 334. The coupling mechanism includes a rotatable chuck 342, a rotational hub 340, a fixed fiber coupled light source bundle 344, and an optical coupler 346. The rotatable chuck 342 and rotational hub 340 couple the proximal end 314 of the imaging probe 310 to the housing 330. The light source bundle 344 optically couples to the illuminating optical fibers 316 to provide infrared light from the infrared light source 332 to the illuminating optical fibers 316. The optical coupler 346 couples to the imaging optical fibers 318 to receive and transmit light from the imaging optical fibers 318 to the infrared light detector 334.

The imaging probe 310 extends through the rotatable chuck 342 and the rotational hub 340 such that the proximal end 314 of the imaging probe 310 extends from the rotational hub 340. The imaging probe 310 is fixed to the rotatable chuck 342 and the rotational hub 340 to enable the imaging probe 310 to be rotated relative to the housing 330 about the longitudinal axis 322 of the imaging probe 310. Thus, the bundle of illuminating optical fibers 316 and the bundle of imaging optical fibers 318 are free to rotate about the longitudinal axis 322, while the fixed fiber coupled light source bundle 344 and optical coupler 346 remain fixed.

The fixed fiber coupled light source bundle 344 and the bundle of illuminating optical fibers 316 form a contact fit when the imaging probe 310 and the housing 330 are coupled together. Similarly, the optical coupler 346 and the bundle of imaging optical fibers 318 form a contact fit when the imaging probe 310 and the housing 330 are coupled together. The coupling mechanism is configured to provide a “quick release” ability to decouple the imaging probe 310 from the housing 330.

The bundle of imaging optical fibers 318 may protrude past the bundle of illuminating optical fibers 316 by distance 348 in a direction of the longitudinal axis 322. In other words, the bundle of illuminating optical fibers 316 may be recessed with respect to the imaging optical fibers 318 by distance 348 in a direction of the longitudinal axis 322. This arrangement may assist the imaging probe 310 to rotate in place while maintaining contact with the infrared light source 332 and infrared light detector 334. This arrangement may further assist in inhibiting the bundle of illuminating optical fibers 316 and the bundle of imaging optical fibers 318 from tangling during rotation.

The infrared light detector 334 may include optics to transmit reflected light from the imaging optical fibers 318 onto a sensing surface. In some examples, the optics may include one or more achromatic doublets to increase convergence of the reflected light for receipt by the sensing surface without wavelength shifting the reflected light.

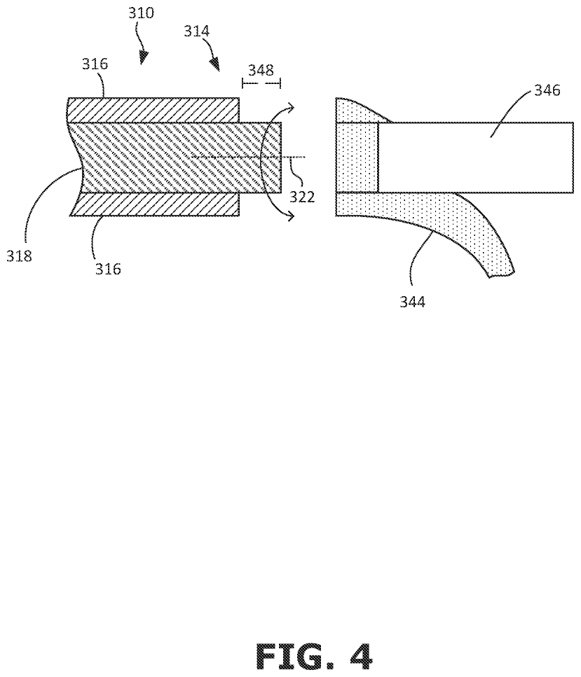

FIG. 4 depicts a schematic diagram of the proximal end 314 of the imaging probe 310 decoupled from the housing 330, showing rotation of the imaging probe 310 about the longitudinal axis 322.

FIG. 5 depicts a plan view of another example imaging probe 510, wherein the imaging probe 510 includes a guidewire sheathing. The imaging probe 510 is substantially similar to the imaging probe 110 of the device 100 with like components having like numbers, however in a “500” series rather than a “100” series. With reference to FIG. 5, the imaging probe 510 includes a distal end 512, a proximal end 514, a bundle of illuminating optical fibers 516 and a bundle of imaging optical fibers 518. For further description of the above elements of the imaging probe 510 the description of the imaging probe 110 of FIG. 1 may be referenced. For sake of clarity, only the differences between the imaging probe 510 and the imaging probe 110 will be described in detail.

In contrast to the imaging probe 110, the imaging probe 510 further includes a sheathing 524 having a leading end portion 550 near the distal end 512, a trailing end portion 554 near the proximal end 514, and a medial portion 552 between the leading end portion 550 and the trailing end portion 554. The imaging probe 110, including the guidewire sheathing, may have a diameter between about 14 thousandths of an inch (0.014″) and about 38 thousandths of an inch (0.038″)..

The leading end portion 550 may include a shapeable coil sheathing portion for flexibility at the distal end 512 of the imaging probe 510 to be shaped in a desired manner, such as to conform to the contours of a particular vasculature. The shapeable coil may include a platinum tungsten or Nitinol tip coil, and may be a close wound coil. In some examples, the leading end portion 550 may include a shapeable memory polymer, for flexibility to be shaped in a desired manner.

The medial portion 552 may include a non-shapeable coil sheathing portion for structural stability with flexibility for the medial portions of the imaging probe 510. The non-shapeable coil may include stainless steel, and may be a close wound coil.

The trailing end portion 554 may include a hypotube portion for structural stability and pushability near the proximal end 514 of the imaging probe 510. The hypotube may include stainless steel such as stainless steel, an alloy, or another suitable rigid material.

In some examples, the imaging probe 510 may have a length from the distal end 512 to the proximal end 514 of about 1500 mm. The leading end portion 550 may have a length of about 150 mm, the medial portion 552 may have a length of about 200 mm, and the trailing end portion 554 may have a length of about 1150 mm.

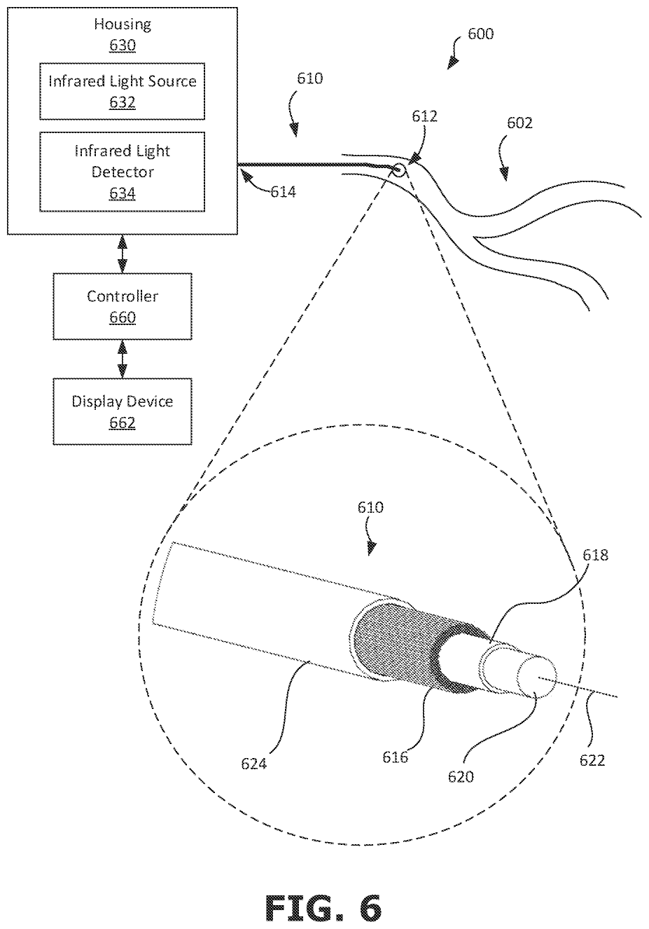

FIG. 6 depicts a schematic diagram of another example device 600 for imaging vasculature. The device 600 is substantially similar to the device 100 with like components having like numbers, however in a “600” series rather than a “100” series. With reference to FIG. 6, the device 600 hence includes an imaging probe 610 having a distal end 612 and proximal end 614, a bundle of illuminating optical fibers 616 and a bundle of imaging optical fibers 618 for imaging vasculature 602, lens 620, optical direction 622, controller 660, and conduit 624. The device 600 further includes an imaging device 630 having an infrared light source 632 and an infrared light detector 634. For further description of the above elements of the device 600, the description of the device 100 of FIG. 1 may be referenced. For sake of clarity, only the differences between the device 600 and the device 100 will be described in detail.

In contrast to the device 100, the device 600 further includes a display device 662 coupled to the controller 660. The display device 662 is to display an image generated by the infrared light detector 634. The display device 662 may be a remote display device. The controller 660 may transmit images of the vasculature 602 to the display device 662 for rendering thereon. The display device 662 may include any suitable display device, including a cathode ray tube display, a liquid crystal display, an organic liquid crystal display, a light emitting diode display, and the like.

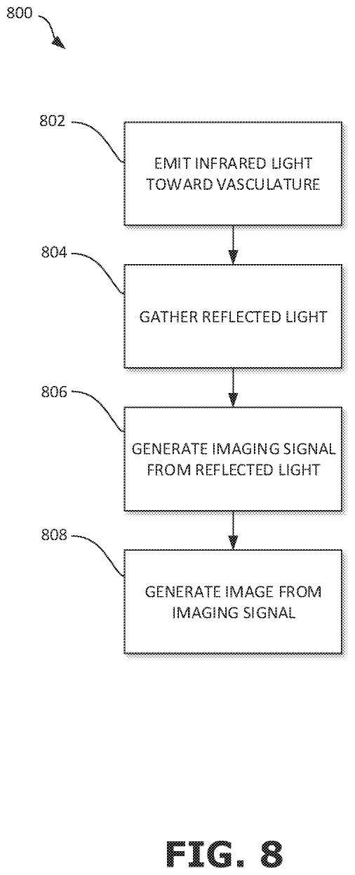

FIG. 8 is a flowchart of an example method 800 for imaging vasculature. The method 800 is one way in which vasculature may be imaged. It is to be emphasized, however, that the blocks of method 800 need not be performed in the exact sequence as shown. Further, the method 800 may be performed by a device as described above such as device 100, 300, or 600. For sake of clarity, the method 800 has been described with reference to the device 100, but this is not limiting, and the method 800 may be performed by other devices.

At block 802, infrared light is emitted from an imaging probe 110 toward the vasculature 102 through blood. At block 804, reflected light is gathered from the vasculature 102 through the blood. At block 806, an imaging signal is generated from the reflected light. At block 808, an image of the vasculature 102 is generated from the imaging signal. The image may be generated according to a ballistic imaging, gated imaging, structured light, polarizing light imaging technique, and the like, as described above.

In some examples, the method 800 may include a ballistic photon imaging process, wherein the imaging probe 110 includes an angle gate filter. In a ballistic photon imaging process, the method 800 includes filtering scattered photons scattered by the blood from the reflected light and collecting ballistic photons from the reflected light. The imaging signal may be generated using the ballistic photons.

In some examples, the method 800 may include a gated imaging process, wherein the infrared light detector 134 includes an infrared camera having a shutter. In a gated imaging process, the method 800 further includes timing the shutter to block scattered photons scattered by the blood from being received by the infrared camera and to allow ballistic photons to be received by the infrared camera. The imaging signal may be generated using the ballistic photons.

In some examples, the method 800 may include a structured light imaging process to generate three-dimensional model of the vasculature 102. In a structured light imaging process, the infrared light emitted from the imaging probe 110 includes a pattern for projection onto the vasculature 102. To generate a three-dimensional model of the vasculature 102 according to a structured light imaging process, the method 800 includes moving the imaging probe 110 through the vasculature 102, generating a plurality of imaging signals from the reflected light as the imaging probe 110 is moved through the vasculature 102, and generating a three-dimensional model of the vasculature 102 from the plurality of imaging signals.

In some examples, the method 800 may include a polarizing light imaging process. In a polarizing light imaging process, the device 100 includes polarization filter. In a polarizing light imaging process, the method 800 includes filtering the reflected light to remove polarized light from the reflected light. The imaging signal may be generated using unpolarized light.

For example, in operation, the imaging probe 110 may be inserted into the vasculature 102. For example, a physician may insert the distal end 112 of the imaging probe 110 into vasculature 102 of a patient. The physician may then push the imaging probe 110 through the vasculature 102 to a desired location. During navigation, the controller 160 may control the infrared light source 132 to provide effective wavelengths of infrared light to penetrate blood to the bundle of illuminating optical fibers 116 of the imaging probe 110. The infrared light travels along the bundle of illuminating optical fibers 116 from the proximal end 114 to the distal end 112 to provide infrared light to the vasculature 102. Light reflected off the vasculature 602 may be collected by the bundle of imaging optical fibers 118 for imaging.

In some examples, images may be captured and displayed via a display similar to the display device 662 in real-time to assist with navigation of the imaging probe 110.

In some examples, an additional medical device such as a catheter may then be brought to the desired location to allow the physician to perform a medical procedure at the desired location. Guidance from the imaging probe 110 which provides real-time imaging and navigation through blood-filled vasculature may enable the medical procedure to be commenced more quickly than if other image guidance techniques were used.

Thus, a device for imaging vascular may be provided which includes an imaging probe to be inserted into a vasculature and to emit infrared light through blood toward the vasculature and to gather reflected from the vasculature for imaging. The imaging device further includes an infrared light source optically coupled to the imaging probe for emission toward the vasculature. The imaging device further includes an infrared light detector optically coupled to the imaging probe to generate an imaging signal from the reflected light, and a controller coupled to the infrared light source and coupled to the infrared light detector to generate an image of the vasculature from the imaging signal. The controller may employ a number of imaging techniques, including ballistic photon imaging techniques, gated imaging techniques, polarizing light imaging techniques, structured light imaging techniques, and the like. The imaging probe may include pushable and trackable sheathing to be navigated through a catheter. The imaging probe may also include guidewire sheathing for navigation through the vasculature. The imaging probe may also be included as part of another catheter device. Thus, such an imaging probe may assist with real-time navigation through vasculature to facilitate quick medical interventions.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” can be construed as X only, Y only, Z only, or any combination of two or more items X, Y, and Z (e.g., XYZ, XY, YZ, XZ, and the like). Similar logic can be applied for two or more items in any occurrence of “at least one . . . ” and “one or more . . . ” language.

The terms “about”, “substantially”, “essentially”, “approximately”, and the like, are defined as being “close to”, for example as understood by persons of skill in the art. In some embodiments, the terms are understood to be “within 10%,” in other embodiments, “within 5%”, in yet further embodiments, “within 1%”, and in yet further embodiments “within 0.5%”.

Persons skilled in the art will appreciate that in some embodiments, the functionality of devices and/or methods and/or processes described herein can be implemented using pre-programmed hardware or firmware elements (e.g., application specific integrated circuits (ASICs), electrically erasable programmable read-only memories (EEPROMs), etc.), or other related components. In other embodiments, the functionality of the devices and/or methods and/or processes described herein can be achieved using a computing apparatus that has access to a code memory (not shown) which stores computer-readable program code for operation of the computing apparatus. The computer-readable program code could be stored on a computer readable storage medium which is fixed, tangible and readable directly by these components, (e.g., removable diskette, CD-ROM, ROM, fixed disk, USB drive). Furthermore, it is appreciated that the computer-readable program can be stored as a computer program product comprising a computer usable medium. Further, a persistent storage device can comprise the computer readable program code. It is yet further appreciated that the computer-readable program code and/or computer usable medium can comprise a non-transitory computer-readable program code and/or non-transitory computer usable medium. Alternatively, the computer-readable program code could be stored remotely but transmittable to these components via a modem or other interface device connected to a network (including, without limitation, the Internet) over a transmission medium. The transmission medium can be either a non-mobile medium (e.g., optical and/or digital and/or analog communications lines) or a mobile medium (e.g., microwave, infrared, free-space optical or other transmission schemes) or a combination thereof.

Persons skilled in the art will appreciate that there are yet more alternative embodiments and modifications possible, and that the above examples are only illustrations of one or more embodiments. The scope, therefore, is only to be limited by the claims appended hereto.

What is claimed is:

1. An imaging device comprising:
a housing;
a display device;
an imaging probe having a proximal end and a distal end, the distal end for insertion into a vasculature, the imaging probe to:
emit infrared light from the distal end of the imaging probe toward the vasculature through blood;
gather reflected light comprising at least a portion of the infrared light reflected from the vasculature through the blood; and
transmit the reflected light to the proximal end of the imaging probe;
an infrared light source disposed in the housing, the infrared light source optically coupled to the proximal end of the imaging probe to provide the infrared light to the imaging probe for emission toward the vasculature, the infrared light source comprising a pattern-generating light source configured to, in a structured light imaging process, project a pattern comprising a grid, or shapes, to generate a three-dimensional representation of the vasculature to display at the display device;
an infrared light detector disposed in the housing, the infrared light detector optically coupled to the proximal end of the imaging probe to receive the reflected light from the imaging probe to generate an imaging signal from the reflected light; and
a controller coupled to the infrared light source and coupled to the infrared light detector to: generate an image of the vasculature from the imaging signal;
and control the infrared light source and the infrared light detector to generate the image according to the structured light imaging process, the image including the three-dimensional representation of the vasculature,
wherein the imaging probe comprises:
a bundle of illuminating optical fibers extending from the proximal end to the distal end, the bundle of illuminating optical fibers to emit the infrared light from the distal end of the imaging probe, the bundle of illuminating optical fibers including coherent illuminating optical fibers to emit the infrared light including the pattern for projection onto the vasculature;
a bundle of imaging optical fibers extending from the proximal end to the distal end, the bundle of imaging optical fibers to gather the reflected light and to transmit the reflected light to the proximal end of the imaging probe; and
optics at the distal end to collect reflected light gathered by the bundle of imaging optical fibers, wherein the optics includes a lens.

2. The imaging device of claim 1, further comprising:
an angle gate filter at the distal end of the imaging probe to filter the reflected light to remove scattered photons from the reflected light;
wherein the controller is further to control the infrared light source and the infrared light detector to generate the image according to a ballistic photon imaging process.

3. The imaging device of claim 1, wherein:
the infrared light detector comprises an infrared camera having a shutter; and
the controller is further to control the infrared light source and the infrared camera to generate the image according to a gated imaging process.

4. The imaging device of claim 1, further comprising:
a polarization filter to filter the reflected light to remove polarized light from the reflected light; and
the controller is further to control the infrared light source and the infrared light detector to generate the image according to a polarizing light imaging process.

5. The imaging device of claim 1, wherein the imaging probe comprises a guidewire sheathing to navigate the imaging probe through the vasculature, and the guidewire sheathing comprises a shapeable coil sheathing portion at about the distal end of the imaging probe, a hypotube portion at about the proximal end of the imaging probe, and a non-shapeable coil sheathing portion between the shapeable coil sheathing portion and the hypotube portion.

6. The imaging device of claim 1, wherein the imaging probe comprises a pushable and trackable sheathing.

7. The imaging device of claim 1, wherein the imaging probe defines a longitudinal axis, and wherein the bundle of imaging optical fibers extends along the longitudinal axis, and the bundle of illuminating optical fibers is arranged in a ring around the bundle of imaging optical fibers.

8. The imaging device of claim 1, wherein the imaging probe defines a longitudinal axis, and wherein the bundle of illuminating optical fibers extends along the longitudinal axis, and the bundle of imaging optical fibers is arranged in a ring around the bundle of illuminating optical fibers.

9. The imaging device of claim 1, wherein the imaging probe comprises a scanning fiber endoscope.

10. The imaging device of claim 1, further comprising a coupling mechanism to reversibly and rotatably couple the proximal end of the imaging probe to the infrared light source and the infrared light detector.

11. A method comprising:
providing an imaging device comprising: a housing; a display device; an imaging probe having a proximal end and a distal end, the distal end for insertion into a vasculature, the imaging probe to: emit infrared light from the distal end of the imaging probe toward the vasculature through blood; gather reflected light comprising at least a portion of the infrared light reflected from the vasculature through the blood; and transmit the reflected light to the proximal end of the imaging probe; an infrared light source disposed in the housing, the infrared light source optically coupled to the proximal end of the imaging probe to provide the infrared light to the imaging probe for emission toward the vasculature, the infrared light source comprising a pattern-generating light source configured to, in a structured light imaging process, project a pattern comprising a grid, or shapes, to generate a three-dimensional representation of the vasculature to display at the display device; an infrared light detector disposed in the housing, the infrared light detector optically coupled to the proximal end of the imaging probe to receive the reflected light from the imaging probe to generate an imaging signal from the reflected light; and a controller coupled to the infrared light source and coupled to the infrared light detector to: generate an image of the vasculature from the imaging signal; and control the infrared light source and the infrared light detector to generate the image according to the structured light imaging process, the image including the three-dimensional representation of the vasculature, wherein the imaging probe comprises: a bundle of illuminating optical fibers extending from the proximal end to the distal end, the bundle of illuminating optical fibers to emit the infrared light from the distal end of the imaging probe, the bundle of illuminating optical fibers including coherent illuminating optical fibers to emit the infrared light including the pattern for projection onto the vasculature; a bundle of imaging optical fibers extending from the proximal end to the distal end, the bundle of imaging optical fibers to gather the reflected light and to transmit the reflected light to the proximal end of the imaging probe; and optics at the distal end to collect reflected light gathered by the bundle of imaging optical fibers, wherein the optics includes a lens;
emitting the infrared light from the imaging probe;
gathering the reflected light;
generating the imaging signal from the reflected light; and
generating the image from the imaging signal.

12. The method of claim 11, wherein the method further comprises:
filtering scattered photons from the reflected light; and
collecting ballistic photons from the reflected light;
wherein the imaging signal is generated using the ballistic photons.

13. The method of claim 11, wherein the reflected light is captured by the infrared camera having a shutter, and wherein the method further comprises:
timing the shutter to block scattered photons from being received by the infrared camera and to allow ballistic photons to be received by the infrared camera;
wherein the imaging signal is generated using the ballistic photons.

14. The method of claim 11, wherein the infrared light emitted from the imaging probe includes the pattern for projection, and wherein the method further comprises:
moving the imaging probe;
generating a plurality of imaging signals from the reflected light as the imaging probe is moved; and
generating a three-dimensional model from the plurality of imaging signals.
